(19) United States
(12) Patent Application Publication
(10) Pub. No.: US 2014/0305462 A1
(43) Pub. Date: Oct. 16, 2014

(54) Powdery Hair Cosmetics

(71) Applicant: Henkel AG & Co. KGaA, Dusseldorf (DE)

(72) Inventors: Thorsten Knappe, Schenefeld (DE); Anna Henschel, Winsen (Luhe) (DE); Bibiane Lange, Sievershuetten (DE)

(21) Appl. No.: 14/364,405

(22) PCT Filed: Nov. 9, 2012
(86) PCT No.: PCT/EP2012/072251

§ 371 Date: Jun. 11, 2014

(30) Foreign Application Priority Data
Dec. 16, 2011 (DE) 10 2011 088 818.7

(51) Int. Cl. A61K 8/02; A61K 8/73; A61Q 5/06; A45D 7/04; A61K 8/81

(52) U.S. Cl. CPC A61K 8/022; A45D 7/04; A61K 8/8182; A61Q 5/06; A61K 8/8152; A61K 8/731
USPC 132/203; 424/70.15; 424/401; 424/70.13

(57) ABSTRACT

Powdery compositions which contain, based on their weight, (a) about 5 to 100% wt. % of at least one powdery compound which is effective as a hair cosmetic agent, (b) 0 to about 25% wt. % of at least one powdery solid as a filler which differs (a) and (c), (c) 0 to about 95% wt. % of at least one auxiliary that is different from (a) and (b) and is selected from emulsifiers and thickeners and the mixtures thereof, with the proviso that about 0.4 g of the powdery composition dissolves in 100 ml water at 35° C. within not more than 5 minutes when mixed in a tumble mixer at 101 revolutions per minute.

### CROSS REFERENCE TO RELATED APPLICATIONS

The present disclosure is a U.S. National Stage entry under 35 U.S.C. §371 based on International Application No. PCT/EP2012/072251, filed Nov. 9, 2012 which was published under PCT Article 21(2) and which claims priority to German Patent Application No. DE 10 2011 088 818.7 filed on Dec. 16, 2011, which is incorporated herein by reference in its entirety.

### TECHNICAL FIELD

The technical field relates to hair care, in particular to powdery hair cosmetics for the temporary shaping of keratin-containing fibers, such as, e.g., temporary hairstyling.

### BACKGROUND

Styling agents for setting keratin-containing fibers have been known for a long time and are used in various configurations for composing, for refreshing, and for fixing hairstyles that for many kinds of hair can only be obtained by using setting active substances. In this regard, hair treatment agents, which provide a permanent or temporary hairstyling, play an important role. Temporary styling, intended to provide a good hold, without compromising the healthy appearance of the hair, such as, for example the gloss, can be obtained by the use of hairsprays, hair waxes, hair gels, setting lotions, etc.

Suitable compositions for temporary hairstyling usually comprise synthetic polymers as the styling component. Preparations comprising a polymer can be applied onto the hair by means of propellants or by a pumping mechanism. Hair gels and hair waxes are however not generally applied directly onto the hair, but rather dispersed in the hair with a comb or by hand.

It is often the case that known forms of temporary styling agents cannot be dosed with sufficient accuracy. Thus, for example, hair gels, hair creams and hair waxes can be dispersed only with difficulty once they have been applied onto the hair. Comparatively large amounts of styling agent are dispensed onto the hair as soon as the comb or the hands, onto which the styling agent was applied, come into contact with the first parts of the hair. In contrast, comparatively little styling agent is worked into parts of the hair which are later reached by the comb or the hands. This means that the users either have to initially apply a large amount of styling agent in order that the parts of the hair reached last also receive sufficient styling agent, or they are obliged to apply the styling agent in a plurality of steps, wherein each of the other parts of the hair are treated. Hair sprays can be dispersed more uniformly onto the hair. However, as the user is not able to visually measure the total amount of the applied styling agent, the danger exists that more styling agent than actually needed could be applied onto the hair.

Powdered cosmetics are known and have already been employed for a long time, for example in the field of hair treatment. Typical examples are make-up powder or eye shadow. The powdery consistency is obtained by employing a powdery carrier material. A metal oxide, for example, e.g. silicon dioxide, can be used as a suitable carrier material. However, the use of metal oxides as the carrier material mostly leads to a metal oxide residue on the keratin-containing fibers. Due to this residue, the keratin-containing fibers treated with this kind of powdered cosmetic lose their natural gloss and become dull. Moreover, the person skilled in the art is aware of powdered or granulated cosmetics from, for example the field of hairdressing products. The hairdresser produces a defined quantity of the hair cosmetic from the powder or granulate by adding an excess of water; the hair cosmetic is then finally used as a cream, gel or lotion on the hair. From the field of temporary styling of keratin-containing fibers, the person skilled in the art is aware of solvent-containing powder in the form of a powder-to-liquid formulation. The published WO 2007/051511 A1 discloses the use of a powdery composition, comprising 50 to 95 wt % of an aqueous solvent, hydrophobic silicon dioxide powder and at least a film-forming and/or setting polymer in the aqueous solvent, for temporarily styling keratinic fibers.

The published WO 2010/054980 A1 discloses the use of a powdery composition containing core-shell particles for temporarily styling keratinic fibers, wherein the shell comprises the particles of a hydrophobic metal oxide powder and the core contains a liquid, aqueous phase. These powdery core-shell particles contain at least one film-forming and/or setting polymer in the form of particles.

In the published WO-A1-2011/076518 there are described powdery compositions that can be converted into a liquid and which contain an active substance adsorbed onto a carrier.

Nowadays the powdery hair cosmetics of the state of the art indeed produce an acceptable hold for the hairstyle. However, the result achieved with these agents could well be improved in regard to gloss and volume as well as in the elasticity of the hold. Furthermore, the known powdery compositions are not compatible with all cosmetic raw materials and have a limited shelf-life.

### SUMMARY

Accordingly, at least one object herein is to provide a powdery hair treatment agent for temporary hairstyling which can be exactly and easily dosed (in particular without clumping when contacted with a little water, and can be well dispersed onto the fibers), as well as provide an improved gloss, volume and an elastic hold to the fibers. In an embodiment, the conservation of the styling result also is not impaired.

### DETAILED DESCRIPTION

It was found that the teaching of the prior art is improved by a powdery composition described below. Powdery compositions with the following requirement profile for application on fibers in the scope of hairstyling keratin-containing fibers are new.

In accordance with a first exemplary embodiment, a powdery compositions contains relative to its total weight
(a) about 5 to 100 wt % of at least one powdery compound, effective as a hair cosmetic agent,
(b) 0 to about 25 wt % of at least one powdery solid as a filler which differs from (a) and (c),
(c) 0 to about 95 wt % of at least one auxiliary that differs from (a) and (b) and is selected from emulsifiers, thickeners as well as their mixtures,
   with the proviso that about 0.4 g of the powdery composition dissolves in 100 mL water at about 35° C. within not more than about 5 minutes, in particular within not more than about 4 minutes, when mixed in a tumble mixer at 101 revolutions per minute.

As used herein, keratinic fibers are understood to mean furs, wool, feathers and particularly human hair.

For purposes herein, compositions are powdery whose particles are freely free-flowing under their own weight (see DIN EN ISO 6186: 1998-08). Particles are small parts of solid objects that exist as a grain (see DIN 66160: 1992-09).

Effective as a hair cosmetic is a substance, if it is intended to be used for this purpose, for cleaning, care or for influencing the appearance or the odor or for imparting impressions of smell; unless it is mainly intended to alleviate or to eliminate illnesses, suffering, physical injuries or pathological conditions.

If a representative of the components (a) additionally fulfils the functional characteristics of the components (c), then in the context herein, it is to be classified as belonging only as a component (a).

For purposes herein, solubility is measured by the following measurement method: In a 250 ml wide-necked screw-top flask thermostatted at 35° C. are placed 100 ml of distilled water at a temperature of 35° C. 0.5 g of the substance to be tested is added. The 250 ml wide-necked screw-top flask is then sealed with a cap, clamped into the clamping device of a tumble mixer (WAB Turbula (type T2F)) and shaken at a rotational speed of 101 revolutions per minute until the substance dissolves. The time measurement begins with the addition of the substance to be tested and ends when the substance dissolves. Five measurements are carried out and the arithmetic mean is calculated.

The powdery compositions contemplated herein can be dosed very easily. They can be very uniformly dispersed in the presence of a small amount of water onto the keratin-containing fibers, i.e., by prior application onto a moist surface of an application aid and/or by application onto moistened hair. The small amount of solvent on the moistened hair or on the moistened surface of an application aid suffices for the focused wetting of the hair fibers with the cosmetic. No formation of lumps occurs.

The powdery composition is preferably characterized in that it comprises, relative to its weight, not more than about 8 wt %, in particular not more than about 5 wt % of a liquid. Said liquid is absorbed by the solid particles of the powder (adsorbent). It is inventively preferred if the powder contemplated herein contains no more than about 5 wt % water as the absorbed liquid.

To improve the hair care, the powdery compositions contemplated herein can preferably comprise at least one care substance of the component (a) as the powdery compound that is effective as the hair cosmetic.

In an exemplary embodiment, the powdery compositions comprise at least one UV filter as the care substance. The inventively suitable UV filters are not generally limited in regard to their structure and their physical properties. Indeed, all UV filters that can be employed in the cosmetic field having an absorption maximum in the UVA (315-400 nm), in the UVB (280-315 nm) or in the UVC (<280 nm) regions are suitable. UV filters having an absorption maximum in the UVB region, especially in the range from about 280 to about 300 nm, are particularly preferred. Those UV filters with a molecular extinction coefficient at the absorption maximum of above about 15 000, particularly above about 20 000, are preferred.

Moreover, it was found that for structurally similar UV filters, the water-insoluble compound exhibits a higher activity than that of water-soluble compounds that differ from them by one or a plurality of additional ionic groups. For purposes herein, water-insoluble UV filters are understood to mean those that dissolve not more than about 1 wt %, especially not more than about 0.1 wt % in water at 20° C. In addition, these compounds should be soluble to at least about 0.1, especially to at least about 1 wt % in conventional cosmetic oil components at room temperature. Accordingly, the use of water-insoluble UV filters can be inventively preferred.

Two preferred UV filters containing cationic groups are the commercially available compounds cinnamidopropyltrimethylammonium chloride (Incroquat® UV-283) and dodecyldimethylaminobenzamidopropyldimethylammonium tosylate (Escalol® HP 610).

Of course, a combination of a plurality of UV filters can also be used. In the context of this embodiment, the combination of at least one water-insoluble UV filter with at least one UV filter containing a cationic group is preferred. The UV filters are usually comprised in the powdery compositions contemplated herein in amounts of about 0.01 to about 5 wt %, relative to the powdery composition. Quantities of about 0.1 to about 2.5 wt % are preferred.

In an exemplary embodiment, the powdery compositions comprise a vitamin, a provitamin, or a vitamin precursor as well as one of their derivatives as an additional care substance. Such vitamins, provitamins and vitamin precursors are preferred that are normally classified in the groups A, B, C, E, F and H.

The compound (3aS,4S,6aR)-2-oxohexahydrothienol[3,4-d]imidazole-4-valeric acid denotes Vitamin H, for which the trivial name biotin has become accepted. Biotin is a quite particularly preferred vitamin. Surprisingly, the restructuration of the fibers could be improved, a structural stabilization achieved and the irritation potential significantly reduced by adding biotin to the preparations contemplated herein. In an embodiment, biotin is present in the powdery compositions in amounts of about 0.0001 to about 2.0 wt %, in particular in amounts of about 0.001 to about 0.2 wt %, each relative to the powdery composition. The vitamin C also is quite particularly preferred.

Panthenol, pantolactone, pyridoxine, derivatives of pyridoxine, nicotinamide, biotin and mixtures thereof are particularly preferred care substances.

In another exemplary embodiment, the powdery compositions comprise at least one plant extract as the care substance. Usually, these extracts are manufactured by extraction of the whole plant. In individual cases, however, the extracts can be produced solely from blossoms and/or leaves of the plant.

Extracts from Moringa Olifeira, green tea, oak bark, stinging nettle, hamamelis, hops, chamomile, burdock root, hawthorn, linden flowers, almonds, aloe vera, coconut, mango, apricot, lime, wheat, kiwi, melon, orange, grapefruit, sage, rosemary, birch, lady's smock, common yarrow, rest-harrow, meristem, ginseng and ginger are particularly preferred. Extracts of Moringa Olifeira, green tea, almond, aloe vera, coconut, mango, apricot, lime, wheat, kiwi and melon are quite particularly suitable. The extraction composition used to prepare the cited plant extracts can be water and alcohols as well as their mixtures. Exemplary preferred alcohols are lower alcohols such as ethanol and isopropanol, but particularly polyhydric alcohols such as ethylene glycol and propylene glycol, both as the sole extraction agent as well as in aqueous mixtures. Plant extracts based on water/propylene glycol in the ratio of about 1:10 to about 10:1 have proven particularly suitable. The plant extracts can be used in pure as well as in diluted form. When they are used in diluted form, they normally comprise ca. 2-80 wt % active substance and the solvent is the extraction agent or mixture of extraction agents used for their extraction. In addition, it can be preferred to employ mixtures of a plurality, particularly two different plant extracts in the powdery compositions contemplated herein.

In an exemplary embodiments, the powdery compositions comprise protein hydrolysate and/or one of its derivatives as the care substance. Protein hydrolysates are product mixtures obtained by acid-, base- or enzyme-catalyzed degradation of proteins (albumins). As used herein, the term “protein hydrolysates” is also understood to mean total hydrolysates as well as individual amino acids and their derivatives as well as mixtures of different amino acids. Furthermore, polymers built up from amino acids and amino acid derivatives are understood to be included in the term “protein hydrolysates.” The latter include for example polyalanine, polyasparagine, polyserine etc. Additional examples of usable compounds are L-alanyl-L-proline, polyglycine, glycyl-L-glutamine or D/L-methionine-S-methylsulfonium chloride. Of course, β-amino acids and their derivatives, like 3-alanine, anthranilic acid or hippuric acid, can also be added. The molecular weight of the protein hydrolysates utilizable herein ranges between about 75, the molecular weight of glycine, and about 200 000, preferably the molecular weight is about 75 to about 50 000 and quite particularly preferably about 75 to about 20 000 Dalton. The added protein hydrolysates can be of both vegetal as well as animal or marine or synthetic origin.

Animal protein hydrolysates are, for example, protein hydrolysates of elastin, collagen, keratin, silk and milk albumin, which can also be present in the form of their salts. Such products are marketed, for example, under the trade names Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex), Sericin (Pentapharm) and Kerasol® (Croda). It is preferred to use protein hydrolysates of vegetal origin, e.g. soya-, almond-, pea-, potato- and wheat protein hydrolysates. Such products are available, for example, under the trade names Gluadin® (Cognis), DiaMin® (Diamalt), Lexein® (Inolex) Hydrosoy® (Croda), Hydrolupin® (Croda), Hydrosesame® (Croda), Hydrotritium® (Croda) and Crotein® (Croda). Although it is preferred to add the protein hydrolysates as such, optionally other mixtures containing amino acids can also be added in their place. Naturally, powdering compositions contemplated herein can include all isomeric forms, such as cis-trans isomers, diastereoisomers and chiral isomers. According to the invention, it is also possible to employ a mixture of a plurality of protein hydrolysates.

In a further exemplary embodiment, the powdery compositions contain ectoine or ectoine derivatives, allantoin, taurine or bisabolol as the care substance.

The term amino acid as used herein is understood to mean the stereoisomeric forms, e.g. D- and L-forms, of the following compounds: asparagine, arginine, aspartic acid, glutamine, glutamic acid, β-alanine, γ-amino butyrate, N_{ε}-acetyl lysine, N_{δ}-acetyl ornithine, N_{γ}-acetyl diamino butyrate, N_{α}-acetyl diamino butyrate, histidine, isoleucine, leucine, methionine, phenylalanine, serine, threonine and tyrosine. L-Amino acids are preferred. Amino acid residues are derived from the corresponding amino acid. The following amino acid residues are preferred:

Gly, Ala, Ser, Thr, Val, β-Ala, γ-amino butyrate, Asp, Glu, Asn, Aln, N_{ε}acetyl lysine, N_{δ}-acetyl ornithine, N_{γ}-acetyl diamino butyrate, N_{α}acetyl diamino butyrate. The abbreviated form of the amino acids conforms to the generally accepted notation. The di- or tripeptide residues are acid amides in their chemical nature and decompose on hydrolysis into 2 or 3 amino acids. The amino acids in the di- or tripeptide residue are bonded together through amide linkages. The powdery compositions preferably comprise these active substances in amounts of about 0.001 to about 2, in particular in amounts of about 0.01 to about 0.5 wt %, each relative to the total weight of the powdery composition.

Particularly preferred powdery substances that are effective as a hair cosmetic are selected from at least one representative of the group that consists of solid, film-forming compounds. Particularly preferred solid, film-forming compounds are selected from at least one compound of the group that is formed from setting polymers, waxes and film-forming polymers, in particular from at least one compound from the group that is formed from setting polymers and waxes.

Polymers as used herein are understood to mean compounds that are constructed from a plurality of molecules, in which a type or a plurality of types of atoms or atom groupings (so-called constitutive units, basic building blocks or repeat units) are repeatedly strung together and which possess a molecular weight of at least about 10 000 g/mol. The polymers are obtained by polyreaction that may occur synthetically or naturally.

Setting polymers contribute to the hold and/or to the creation of the hair volume and the hair body of the whole hairstyle. These polymers are also film-forming polymers at the same time and therefore in general are typical substances for hair styling treatment agents such as hair sets, hair foams, hair waxes, and hair sprays. The film formation can be in completely selected areas and bond only some fibers together.

Film-forming compounds or film-forming polymers are understood to mean those compounds/polymers that on drying leave a continuous film on the skin, the hair or the nails. These types of film-former can be used in the widest variety of cosmetic products such as, for example make up masks, make up, hair sets, hair sprays, hair gels, hair waxes, hair conditioners, shampoos or nail varnishes.

Film-forming compounds or film-forming polymers are further understood to mean those compounds or polymers that, when used in concentrations of about 0.01 to about 20 wt % in aqueous, alcoholic or aqueous alcoholic solution, are able to precipitate out a transparent film on the hair.

In an exemplary embodiment, the setting polymers are present in an amount of about 2.0 wt % to about 95 wt %, particularly about 5.0 wt % to about 95.0 wt %, quite particularly preferably about 7.5 wt % to about 95.0 wt %, each relative to the weight of the powdery composition.

In the context of a particularly preferred embodiment, the powdery compositions comprise at least one non-ionic setting polymer as the powdery compound that is effective as a hair cosmetic agent. In an embodiment, the powdery composition contemplated herein comprises the non-ionic setting polymers in an amount of about 2.0 wt % to about 95 wt %, particularly about 5.0 wt % to about 95.0 wt %, quite particularly preferably about 7.5 wt % to about 95.0 wt %, each relative to the weight of the powdered composition according to the invention.

The non-ionic setting polymers are again preferably selected from at least one polymer of the group that consists of
homopolymers and non-ionic copolymers of N-vinyl pyrrolidone,
non-ionic copolymers of isobutene,
non-ionic copolymers of maleic anhydride.

Suitable polyvinyl pyrrolidones are for example commercial products such as Luviskol® K 90 or Luviskol® K 85 from BASF SE. A suitable polyvinyl acetate is marketed for example as an emulsion under the trade name Vinac® by Air Products.

Powdery compositions contemplated herein which comprise at least one polymer as the non-ionic setting polymer selected from the group consisting of
copolymers of maleic anhydride and methyl vinyl ether,
polyvinyl pyrrolidone,
copolymers of N-vinyl pyrrolidone and vinyl esters of carboxylic acids containing 2 to 18 carbon atoms, especially from N-vinyl pyrrolidone and vinyl acetate,
   or mixtures of these polymers, are quite particularly preferred. In this regard, those powdery compositions are again preferred which comprise at least one polymer as the non-ionic setting polymer selected from the group consisting of
polyvinyl pyrrolidone,
copolymers of N-vinyl pyrrolidone and vinyl esters of carboxylic acids containing 2 to 18 carbon atoms, especially from N-vinyl pyrrolidone and vinyl acetate,
   or mixtures of these polymers.

If copolymers of N-vinyl pyrrolidone and vinyl esters of carboxylic acids containing 2 to 18 carbon atoms, especially from N-vinyl pyrrolidone and vinyl acetate are employed, it is again preferred if the molar ratio of the resulting structural units of the N-vinyl pyrrolidone monomer to the resulting structural units of the vinyl esters of carboxylic acids containing 2 to 18 carbon atoms (especially vinyl acetate) monomer is in the range of about 20 to 80 to about 80 to 20, especially from about 30 to 70 to about 60 to 40.

Suitable copolymers of vinyl pyrrolidone and vinyl acetate are available for example under the trade names Luviskol® VA 37, Luviskol® VA 55, Luviskol® VA 64 and Luviskol® VA 73 from BASF SE.

In an embodiment, the powdery compositions contemplated herein comprise at least one cationic setting polymer as the powdery compound that is effective as a hair cosmetic agent. Cationic polymers are understood to mean polymers that, in their main chain and/or side chain, possess groups that can be “temporarily” or “permanently” cationic. “Permanently cationic” refers herein to those polymers, which independently of the pH of the composition, have a cationic group. These are generally polymers which comprise a quaternary nitrogen atom in the form of an ammonium group, for example. Preferred cationic groups are quaternary ammonium groups. In particular, those polymers, in which the quaternary ammonium groups are bonded through a C₁₋₄ hydrocarbon group to a polymer backbone formed from acrylic acid, methacrylic acid or their derivatives, have proved to be particularly suitable.

An example of a suitable cationic setting polymer is at least one cationic setting polymer that comprises at least one structural element of the Formula (M9) and optionally additionally at least one structural element of the Formula (M10)

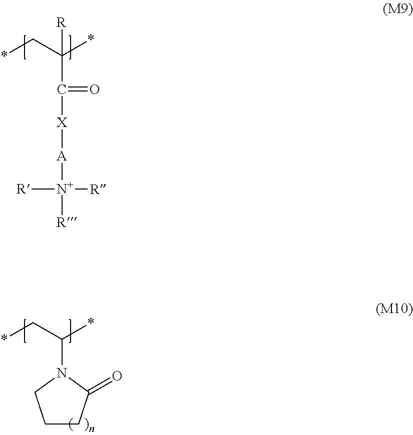

in which
R stands for a hydrogen atom or a methyl group,
R′, R″ and R′″ stand independently of one another for a (C₁ to C₃₀) alkyl group,
X stands for an oxygen atom or an NH group,
A stands for an ethane-1,2-diyl group or a propane-1,3-diyl group,
n means 1 or 3.
To compensate for the positive polymer charge, all possible physiologically acceptable anions may be used, such as for example chloride, bromide, hydrogen sulfate, methyl sulfate, ethyl sulfate, tetrafluoroborate, phosphate, hydrogen phosphate, dihydrogen phosphate or p-toluene sulfonate, triflate.

At least one of the following compounds is preferably employable as the said cationic setting polymer
homopolymers of 2-(N,N,N-trimethylammoniumethyl) methacrylate (in particular the chloride with the INCI name Polyquaterium-37, for example under the trade name Ultragel 300 (BASF SE).
copolymers of dimethylaminoethyl methacrylate, quaternized with diethyl sulfate, with vinyl pyrrolidone having the INCI name Polyquaternium-11 under the trade names Gafquat® 440, Gafquat® 734, Gafquat® 755 (each from ISP) and Luviquat PQ 11 PN (BASF SE),
copolymer of N-vinyl pyrrolidone, N-vinyl caprolactam, N-(3-dimethylaminopropyl)methacrylamide and 3-(methacryloylamino)propyl-lauryl-dimethylammonium chloride (INCI name: Polyquaternium-69) that is commercialized for example under the trade name Styleze W 10 or W 20 (10 or 20 wt % active substance in ethanol-water mixture) by the ISP Company.
copolymer of N-vinyl pyrrolidone, N-vinyl caprolactam, N-(3-dimethylaminopropyl)methacrylamide and 3-(methacryloylamino)propyl-lauryl-dimethylammonium chloride (INCI name: Polyquaternium-69) that is commercialized for example by the ISP Company under the trade name Aquastyle® 300 (28-32 wt % active substance in ethanol/water mixture).

In an exemplary embodiment, those cationic setting copolymers that comprise at least one structural element of the Formula (M11) additionally serve as usable cationic polymers

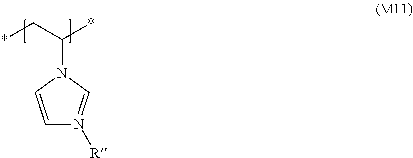

in which R″ stands for a (C₁ to C₄) alkyl group, in particular a methyl group, and additionally possess at least one further cationic and/or non-ionic structural element. To compensate for the positive polymer charge, all possible physiologically acceptable anions may be used, such as for example chloride, bromide, hydrogen sulfate, methyl sulfate, ethyl sulfate, tetrafluoroborate, phosphate, hydrogen phosphate, dihydrogen phosphate or p-toluene sulfonate, triflate.

It is again preferred when at least one copolymer (c1) is comprised as the additional cationic setting polymer, wherein said copolymer (c1) additionally contains a structural element of the Formula (M6) besides at least one structural element of the Formula (M11)

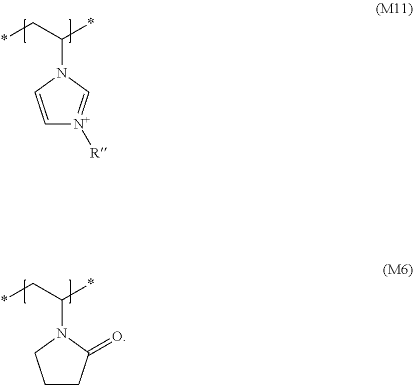

in which R″ stands for a (C₁ to C₄) alkyl group, in particular a methyl group. To compensate for the positive polymer charge of the copolymer (c1), all possible physiologically acceptable anions may be used, such as for example chloride, bromide, hydrogen sulfate, methyl sulfate, ethyl sulfate, tetrafluoroborate, phosphate, hydrogen phosphate, dihydrogen phosphate or p-toluene sulfonate, triflate.

Cationic setting polymers that are quite particularly preferred as the copolymers (c1) contain about 10 to about 30 mol %, preferably about 15 to about 25 mol % and particularly about 20 mol % of structural units in accordance with Formula (M11) and about 70 to about 90 mol %, preferably about 75 to about 85 mol % and particularly about 80 mol % of structural units in accordance with Formula (M6).

In this regard it is particularly preferred when the copolymers (c1) comprise maximum about 5 wt %, preferably maximum about 1 wt % of polymer units that trace back to the incorporation of other monomers, in addition to polymer units that result from the incorporation of the cited structural units in accordance with Formula (M11) and (M6) into the copolymer. The copolymers (c1) are preferably exclusively constructed of structural units of the Formula (M11) with R″=methyl and (M6).

If a chloride ion is used to compensate the positive charge of the polymer of Formula (PoIy1), then these N-methyl vinyl imidazole/vinyl pyrrolidone copolymers are named according to INCI nomenclature as Polyquaternium-16 and are available for example from BASF SE under the trade names Luviquat® Style, Luviquat® FC 370, Luviquat® FC 550, Luviquat® FC 905 and Luviquat® HM 552. If a methosulfate ion is used to compensate the positive charge of the polymer of Formula (PoIy1), then these N-methyl vinyl imidazole/vinyl pyrrolidone copolymers are named according to INCI nomenclature as Polyquaternium-44 and are available for example from BASF SE under the trade name Luviquat® UltraCare.

In another embodiment, in addition to the copolymer(s) (c1) or instead of it or them, the inventive powdered compositions include copolymers (c2) that starting from the copolymer (c1) possess structural units of the Formula (M7) as the additional structural units

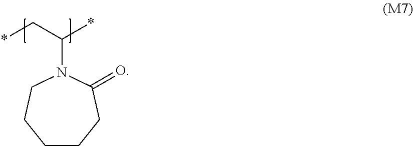

Further particularly preferred powdered compositions are accordingly characterized in that they comprise as the cationic setting polymer at least one copolymer (c2) that comprises at least one structural unit according to Formula (M11-a) and at least one structural unit according to Formula (M-6) and at least one structural unit according to Formula (M-7)

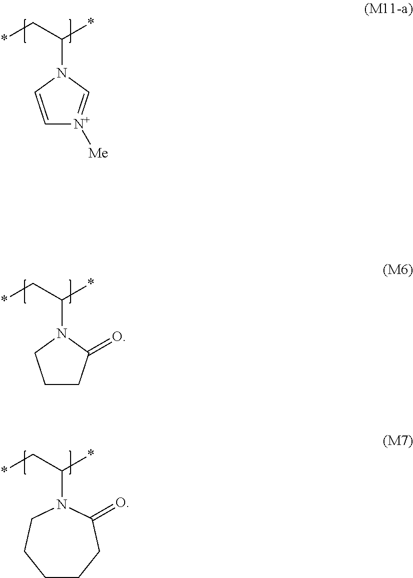

Also in this regard it is particularly preferred when the copolymers (c2) comprise, in addition to polymer units that result from the incorporation of the cited structural units in accordance with Formula (M11-a), (M6) and (M7) into the copolymer, maximum about 5 wt %, preferably maximum about 1 wt % of polymer units that trace back to the incorporation of other monomers. The copolymers (c2) are preferably exclusively constructed from structural units of the Formulas (M11-a), (M6) and (M7).

To compensate for the positive polymer charge of the component (c2), all possible physiologically acceptable anions may be used, such as for example chloride, bromide, hydrogen sulfate, methyl sulfate, ethyl sulfate, tetrafluoroborate, phosphate, hydrogen phosphate, dihydrogen phosphate or p-toluene sulfonate, triflate. If a methosulfate ion is used to compensate the positive charge of the polymer of Formula (PoIy2), then these N-methyl vinyl imidazole/vinyl pyrrolidone/vinyl caprolactam copolymers are named according to INCI nomenclature as Polyquaternium-46 and are available from for example BASF SE under the trade name Luviquat® Hold.

Quite particularly preferred copolymers (c2) comprise about 1 to about 20 mol %, preferably about 5 to about 15 mol % and particularly about 10 mol % of structural units in accordance with Formula (M11-a) and about 30 to about 50 mol %, preferably about 35 to about 45 mol % and particularly about 40 mol % of structural units in accordance with Formula (M6) and 40 to about 60 mol %, preferably about 45 to about 55 mol % and particularly about 60 mol % of structural units in accordance with Formula (M7).

In an embodiment, in addition to the copolymer(s) (c1) and/or (c2) or in its or their place the powdered compositions contemplated herein can also comprise copolymers (c3) as the setting cationic polymer which possess as the structural units structural units of the Formulas (M11-a) and (M6), as well as additional structural units from the group of the vinyl imidazole units and further structural units from the group of the acrylamide and/or methacrylamide units.

Further particularly preferred powdery compositions comprise as the additional cationic setting polymer at least one copolymer (c3) that comprises at least one structural unit according to Formula (M11-a) and at least one structural unit according to Formula (M6) and at least one structural unit according to Formula (M10) and at least one structural unit according to Formula (M12)

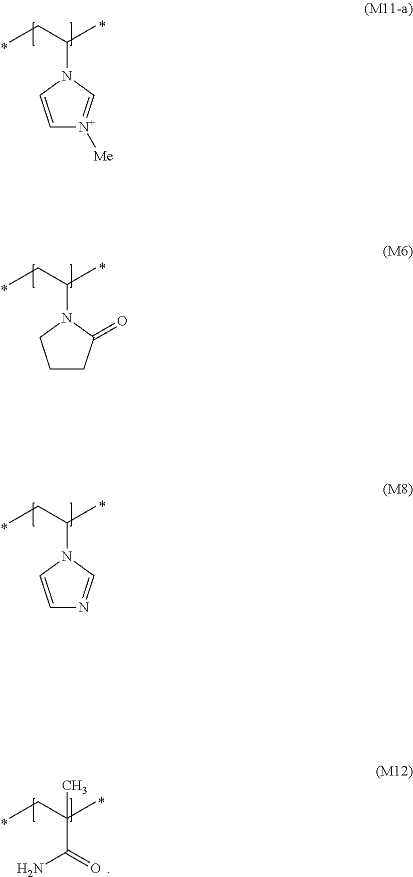

Also in this regard it is particularly preferred when the copolymers (c3) comprise, in addition to polymer units that result from the incorporation of the cited structural units in accordance with Formula (M11-a), (M6), (M8) and (M12) into the copolymer, maximum about 5 wt %, preferably maximum about 1 wt % of polymer units that trace back to the incorporation of other monomers. The copolymers (c3) are preferably exclusively constructed from structural units of the Formulas (M11-a), (M6), (M8) and (M12).

To compensate for the positive polymer charge of the component (c3), all possible physiologically acceptable anions may be used, such as for example chloride, bromide, hydrogen sulfate, methyl sulfate, ethyl sulfate, tetrafluoroborate, phosphate, hydrogen phosphate, dihydrogen phosphate or p-toluene sulfonate, triflate. If a methosulfate ion is used to compensate the positive charge of the polymer of Formula (PoIy3), then these N-methyl vinyl imidazole/vinyl pyrrolidone/vinyl imidazole/methacrylamide copolymers are named according to INCI nomenclature as Polyquaternium-68 and are available from for example BASF SE under the trade name Luviquat® Supreme.

Quite particularly preferred copolymers (c3) contain about 1 to about 12 mol %, preferably about 3 to about 9 mol % and particularly about 6 mol % of structural units in accordance with Formula (M11-a) and about 45 to about 65 mol %, preferably about 50 to about 60 mol % and particularly about 55 mol % of structural units in accordance with Formula (M6) and about 1 to about 20 mol %, preferably about 5 to about 15 mol % and particularly about 10 mol % of structural units in accordance with Formula (M8) and about 20 to about 40 mol %, preferably about 25 to about 35 mol % and particularly about 29 mol % of structural units in accordance with Formula (M12).

Further particularly preferred powdery compositions comprise as the cationic setting polymer at least one copolymer (c4) that comprises at least one structural unit according to Formula (M6), at least one structural unit according to Formula (M13), at least one structural unit according to Formula (M8) and at least one structural unit according to Formula (M11),

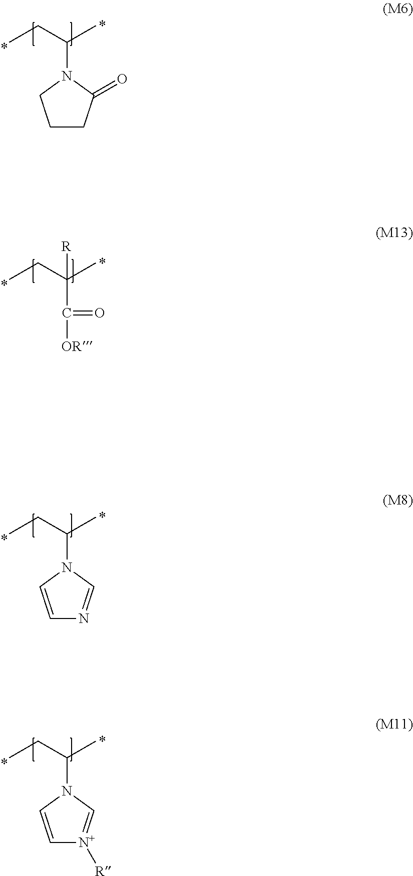

in which
R″ stands for a (C₁ to C₄) alkyl group, particularly a methyl group,
R stands for a hydrogen atom or a methyl group,
R′″ stands for a hydrogen atom or one equivalent of a physiologically acceptable cation. To compensate for the positive charge of the structural unit (IV), all possible physiologically acceptable anions may be used, such as for example chloride, bromide, hydrogen sulfate, methyl sulfate, ethyl sulfate, tetrafluoroborate, phosphate, hydrogen phosphate, dihydrogen phosphate or p-toluene sulfonate, triflate. Exemplary (C₁ to C₄) alkyl groups are methyl, ethyl, propyl, isopropyl, butyl, sec-butyl, isobutyl, tert-butyl. In an embodiment, the charge density of the cationic charge of said copolymer is about 0.8 meq/g at a pH of pH 7.

In an embodiment, R in Formula (M13) stands for a methyl group. In another embodiment, R′″ in Formula (M13) stands for a hydrogen atom. In yet another embodiment, R″ in Formula (M11) stands for a methyl group.

An particularly preferred copolymer contains at least one structural unit of Formula (M6), at least one structural unit of Formula (M13-a), at least one structural unit of Formula (M8) and at least one structural unit of Formula (M11-a),

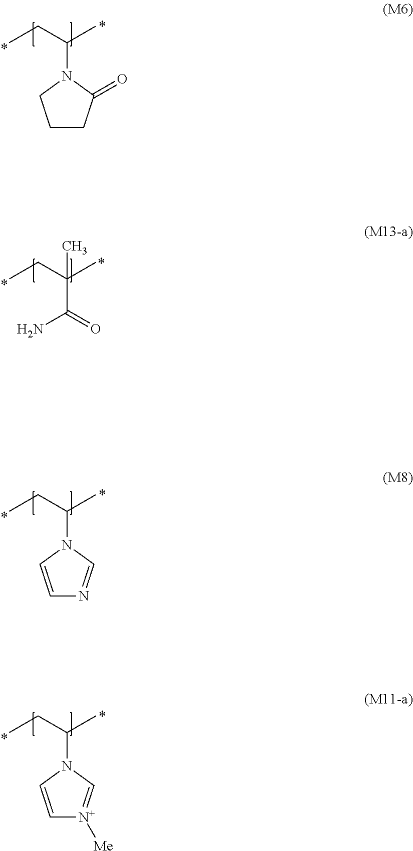

The agents contemplated herein quite particularly preferably comprise a copolymer of N-vinyl pyrrolidone, N-vinyl imidazole, N-methyl-N′-vinylimidazolium chloride and methacrylic acid as said copolymer. It is most preferred if the copolymer named with the INCI name Polyquaternium-86 is the copolymer. INCI is the abbreviation for International Nomenclature of Cosmetic Ingredients. Such copolymers are marketed for example by BASF SE under the trade name Luvigel® Advanced in the form of a white powder.

In an exemplary embodiment, powdery compounds that are effective as a hair cosmetic contain a cationic polymer with at least one structural element of the above Formula (M11-a) selected from
vinyl pyrrolidone/1-vinyl-3-methyl-1H-imidazolium chloride copolymers (such as for example that with the INCI name Polyquaternium-16, sold under the trade names Luviquat® Style, Luviquat® FC 370, Luviquat® FC 550, Luviquat® FC 905 and Luviquat® HM 552 (BASF SE)),
vinyl pyrrolidone/1-vinyl-3-methyl-1H-imidazolium methyl sulfate copolymers (such as for example that with the INCI name Polyquaternium-44 sold under the trade name Luviquat® Care (BASF SE)),
vinyl pyrrolidone/vinyl caprolactam/1-vinyl-3-methyl-1H-imidazolium terpolymer (such as for example that with the INCI name Polyquaternium-46 sold under the trade names Luviquat® Care or Luviquat® Hold (BASF SE)),
vinyl pyrrolidone/methacrylamide/vinyl imidazole/1-vinyl-3-methyl-1H-imidazolium methyl sulfate copolymer (such as for example that with the INCI name Polyquaternium-68 sold under the trade name Luviquat® Supreme (BASF SE)),
copolymer of N-vinyl pyrrolidone, N-vinyl imidazole, N-methyl-N′-vinylimidazolium chloride and methacrylic acid (such as for example that with the INCI name

Polyquaternium-86 sold under the trade name Luvigel® Advanced (BASF SE)) as well as mixtures of these polymers.

Further preferred cationic polymers that can be employed in the powdery compositions are the “temporarily cationic” polymers. These polymers usually comprise an amino group that is present at specific pH values as a quaternary ammonium group and is thus cationic.

Preferred suitable temporarily cationic polymers useful herein are likewise those that possess at least one structural unit of the Formulas (M1-1) to (M1-8)

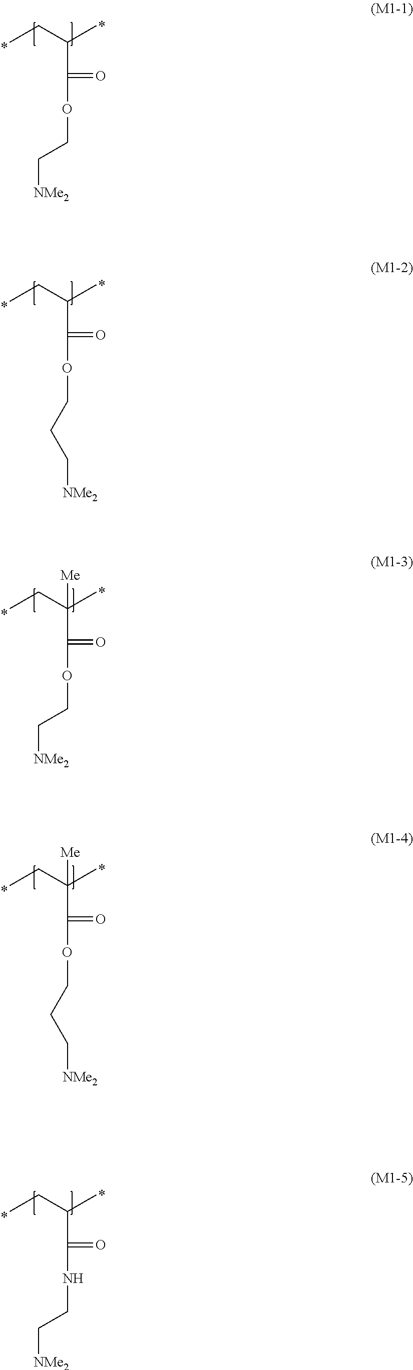

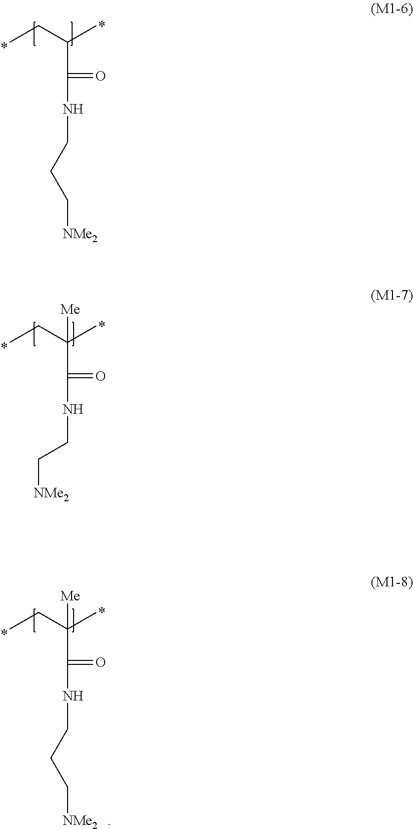

In this regard, those copolymers are again preferred that comprise at least one structural unit of the Formulas (M1-1) to (M1-8) and in addition at least one structural unit of the Formula (M10),

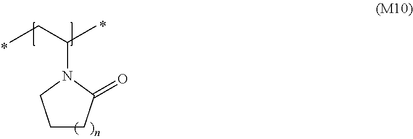

in which
n means 1 or 3.

Here again, the group of the polymers
vinyl caprolactam/vinyl pyrrolidone/dimethylaminoethyl methacrylate copolymer (for example NCI name: Vinyl Caprolactam/PVP/Di-methylaminoethyl Methacrylate Copolymer under the trade name Gaffix® VC 713 (ISP)),
vinyl pyrrolidone/vinyl caprolactam/dimethylaminopropylmethacrylamide copolymer
(for example INCI name: VP/Vinyl Caprolactam/DMAPA Acrylates Copolymer under the trade name Aquaflex® SF-40 (ISP)),
vinyl caprolactam/vinyl pyrrolidone/dimethylaminoethyl methacrylate copolymer (for example as a powder in the form of the commercial product Advantage S with the INCI name: Vinyl Caprolactam/VP/Di-methylaminoethyl Methacrylate Copolymer (ISP)),
vinyl pyrrolidone/dimethylaminopropylmethacrylamide copolymer (for example INCI name: VP/DMAPA Acrylates Copolymer under the trade name Styleze® CC-10 (ISP)), represents the preferred list, from which at least one or more polymers may be selected. In an exemplary embodiment, the powdery composition contemplated herein comprises the cationic polymers in an amount of about 2.0 wt % to about 95 wt %, particularly about 5.0 wt % to about 95.0 wt %, quite particularly preferably about 7.5 wt % to about 95.0 wt %, each relative to the total weight of the powdery composition.

The powdery compositions can also comprise at least one amphoteric setting polymer as the powdery compound that is effective as a hair cosmetic agent. The term amphoteric polymers embraces not only those polymers whose molecule comprises both free amino groups as well as free —COOH or SO₃H groups and which are capable of forming inner salts, but also zwitterionic polymers whose molecule comprises quaternary ammonium groups and —COO⁻ or —SO₃⁻ groups, and those polymers that comprise —COOH or SO₃H groups and quaternary ammonium groups. An example of an amphopolymer which may be useful herein is the acrylic resin obtainable under the designation Amphomer®, which constitutes a copolymer of tert-butylaminoethyl methacrylate, N-(1,1,3,3-tetramethylbutyl)acrylamide as well as two or more monomers from the group consisting of acrylic acid, methacrylic acid and their simple esters. In an exemplary embodiment, the powdered compositions preferably comprise the amphoteric setting polymers in an amount of about 0.1 wt % to about 20.0 wt %, particularly preferably about 0.05 wt % to about 10.0 wt %, each relative to the weight of the total composition. Quantities of about 0.1 to about 5.0 wt % are quite particularly preferred.

In a further embodiment, at least one anionic setting polymer is employed as the powdery compound that is effective as a hair cosmetic. The anionic polymers concern anionic polymers that preferably possess carboxylate and/or sulfonate groups. Exemplary anionic monomers, from which such polymers can be made, are acrylic acid, methacrylic acid, crotonic acid, maleic anhydride and 2-acrylamido-2-methylpropane sulfonic acid. Here, the acidic groups may be fully or partially present as the sodium, potassium, ammonium, mono or triethanolammonium salt.

Within this embodiment, it is preferred to use copolymers of at least one anionic monomer and at least one non-ionic monomer. Regarding the anionic monomers, reference is made to the abovementioned substances. Preferred non-ionic monomers are acrylamide, methacrylamide, acrylic acid esters, methacrylic acid esters, vinyl pyrrolidone, vinyl ethers and vinyl esters.

Preferred anionic copolymers are acrylic acid-acrylamide copolymers and particularly polyacrylamide copolymers with monomers that contain sulfonic acid groups. A particularly preferred anionic copolymer consists of about 70 to about 55 mole % acrylamide and about 30 to about 45 mole % 2-acrylamido-2-methylpropane sulfonic acid, wherein the sulfonic acid group may be fully or partially present as the sodium, potassium, ammonium, mono or triethanolammonium salt. Further preferred employable anionic polymers are selected from the group consisting of:
copolymers of vinyl acetate and crotonic acid (as are marketed for example by CIBA in a 60 wt % conc. dispersion in isopropanol-water as the commercial product Aristoflex® A 60 with the NCI name VA/Crotonates Copolymer),
copolymers of ethyl acrylate and methacrylic acid (as are marketed for example by BASF SE under the trade name Luviflex® Soft with an acid number of 84 to 105 under the INCI name Acrylates Copolymer in a ca. 20 to 30 wt % conc. dispersion in water),
polyurethanes containing at least one carboxylic group (such as for example a copolymer of isophthalic acid, adipic acid, 1,6-hexane diol, neopentyl glycol and isophorone diisocyanate as is marketed by BASF SE under the trade name Luviset® PUR with the NCI name Polyurethane-1).

In an exemplary embodiment, at least one wax is suitable as an powdery compound that is effective as the hair cosmetic. Waxes exhibit a melting point in a range of about 38° C. to about 95° C. at about 1013 mbar. The waxes are preferably selected among vegetal, animal and mineral waxes, preferred waxes being those with a melting point in the range about 50 to about 90° C.

The powdery composition contemplated herein particularly preferably comprises at least one wax selected from beeswax (Cera Alba), carnauba wax, candelilla wax, rice bran wax, Montan wax, paraffin wax, microcrystalline paraffin wax, ozokerite wax and cetyl palmitate.

In an exemplary embodiment, a plurality of waxes also are useful. Thus, an addition of carnauba wax can be used in order to increase the melting point and drop point of another wax and to reduce its tack. A series of wax mixtures, optionally blended with other additives, are correspondingly commercially available. Exemplary employed mixtures are the “Spezialwachs 7686 OE” (a mixture of cetyl palmitate, beeswax, microcrystalline wax and polyethylene with a melting range of 73-75° C.; manufacturer: Kahl & Co), Kahlwax 6240 (Hydrogenated Vegetable Oil) and “Weichceresin® FL 400” (a Vaseline/Vaseline oil/wax mixture with a melting point of 50-54° C.; manufacturer: Parafluid Mineralolgesellschaft).

It has proved particularly preferable if the compositions contemplated herein together with at least one wax additionally comprise at least one emulsifier as the powdery compound that is an effective hair cosmetic. In the context of this embodiment, liquid emulsifiers may also be employed, as long as they afford a powdery compound when blended with the at least one wax, e.g., by absorption.

Preferred emulsifiers are non-ionic and comprise as the hydrophilic group, for example, a polyol group, a polyalkylene glycol ether group or a combination of polyol and polyglycol ether groups. The powdery composition contemplated herein preferably comprises, in addition to the at least one wax, at least one non-ionic emulsifier selected from:
addition products of 2 to 100 mol of ethylene oxide and/or 1 to 5 mol of propylene oxide to saturated or unsaturated, linear or branched fatty alcohols containing 8 to 30 carbon atoms, to fatty acids containing 8 to 30 carbon atoms,
methyl or C₂-C₆ alkyl residue end blocked addition products of 2 to 50 mol ethylene oxide and/or 1 to 5 mol propylene oxide to linear and branched fatty alcohols with 8 to 30 carbon atoms, to fatty acids with 8 to 30 carbon atoms and to alkylphenols with 8 to 15 carbon atoms in the alkyl group, such as, for example, the available types under the sales names Dehydol® LS, Dehydol® LT (Cognis),
C₁₂-C₃₀ fatty acid mono and diesters of addition products of 1 to 30 mol ethylene oxide to glycerin,
addition products of 5 to 60 mol ethylene oxide to castor oil and hydrogenated castor oil,
polyol esters of fatty acids, such as, for example, the commercial product Hydagen® HSP (Cognis) or Sovermol types (Cognis),
alkoxylated triglycerides,
alkoxylated fatty acid alkyl esters of the Formula (E4-I)

R¹CO—(OCH₂CHR²)_{w}OR³ (E4-I)
in which R¹CO stands for a linear or branched, saturated and/or unsaturated acyl residue containing 6 to 22 carbon atoms, R² for hydrogen or methyl, R³ for linear or branched alkyl residues containing 1 to 4 carbon atoms and w for numbers from 1 to 20,
amine oxides,
hydroxy mixed ethers,
sorbitol esters of fatty acids and addition products of ethylene oxide to sorbitol esters of fatty acids such as for example the polysorbates,
sugar esters of fatty acids and addition products of ethylene oxide to sugar esters of fatty acids,
addition products of ethylene oxide to fatty acid alkanolamides and fatty amines,
sugar surfactants of the type alkyl and alkenyl oligoglycosides according to Formula (E4-II),

R⁴O-[G]ₚ (E4-II)
in which R⁴ stands for an alkyl or alkenyl residue containing 4 to 22 carbon atoms, G for a sugar residue containing 5 or 6 carbon atoms and p for numbers from 1 to 10.

In the context of the indicated quantities as the component (c) of the powdery composition contemplated herein, the emulsifiers, in particular the non-ionic emulsifiers, are preferably present in an amount of about 0.5 to about 20.0, in particular about 1.0 to about 15 wt % based on the total weight of the agent.

The powdery composition relative to its weight preferably comprises as the component (b) at least one powdery solid that differs from component (a) and component (c) as the filler in an amount of 0 to about 10 wt %, particularly preferably 0 to about 5 wt %, quite particularly preferably 0 to about 1.0 wt %.

The powdery solids of component (b) are preferably water-soluble. However, it is tolerable to use between 0 and about 5.0 wt % water-insoluble powdery solids as the filler. In this case, the solubility test according to the requirement of claim 1 is carried out without the water-insoluble ingredients of the component (b). For the most optimal solution of the object, the powdery composition contemplated herein is quite particularly preferably free of powdery, water-insoluble solids as the filler. A substance is water-insoluble if at most about 0.1 g of the substance dissolves in 100 mL distilled water at 35° C.

Metal oxides are particularly suitable as the powdery solids of the component (b). These metal oxides can be hydrophilic or hydrophobic. Preferred suitable metal oxides are selected from at least one representative of the group that consists of silicates, aluminum silicates, titanium dioxide, zinc dioxide as well as silicon dioxide. Particularly preferred aluminum silicates (also called alumosilicates) are selected from phyllosilicates, tectosilicates or mixtures thereof. Preferred suitable phyllosilicates are selected from kaolins (here, especially kaolinite, dickite, hallosite as well as nacrite), serpentine, talc, pyrophillite, montmorillonite, quartz, bentonite, mica (here, especially illite, muscovite, paragonite, phlogopite, biotite, lepidolith, margarit, smectite (here especially montmorrilionite, saponite, nontronite, hectorite).

Preferred suitable tectosilicates are selected from feldspar minerals (especially albite, orthoclase, anorthite, leucite, sodalite, hauyne, labradorite, lasurite, nosean, nepheline), zeolites.

Phosphates are likewise suitable as the powdery solids of the component (b), in so far as their use should not be avoided on ecological grounds. Among the many commercially available phosphates, the alkali metal phosphates are well suited and pentasodium or pentapotassium triphosphate (sodium or potassium tripolyphosphate) are particularly preferred.

“Alkali metal phosphates” is the collective term for the alkali metal (more particularly sodium and potassium) salts of the various phosphoric acids, in which metaphosphoric acids (HPO₃)ₙ and orthophosphoric acid H₃PO₄ are differentiated from representatives of higher molecular weight. Examples of such phosphates that may be cited are the pentasodium triphosphate, Na₅P₃O₁₀ (sodium tripolyphosphate) as well as the corresponding potassium salt pentapotassium triphosphate K₅P₃O₁₀ (potassium tripolyphosphate). The sodium potassium tripolyphosphates are again preferably employed.

Further suitable powdery solids of the component (b) are dextrins, for example oligomers or polymers of carbohydrates that can be obtained by the partial hydrolysis of starches. The hydrolysis can be carried out using conventional processes, for example acidic or enzymatic catalyzed processes. The hydrolysis products preferably have average molecular weights in the range 400 to 500 000 g/mol. A polysaccharide with a dextrose equivalent (DE) of 0.5 to 40 and, more particularly, 2 to 30 is preferred, the DE being an accepted measure of the reducing effect of a polysaccharide in comparison with dextrose, which has a DE of 100. Both maltodextrins with a DE between 3 and 20 and dry glucose syrups with a DE between 20 and 37 and also so-called yellow dextrins and white dextrins with higher molecular weights of about 2 000 to about 30 000 g/mol may be used. The oxidized derivatives of such dextrins concern their reaction products with oxidizing agents that are capable of oxidizing at least one alcohol function of the saccharide ring to the carboxylic acid function. Mineral salts of monovalent metal ions, such as for example Na₂SO₄ or sodium chloride, have likewise proved to be suitable.

In an exemplary embodiment, the powdery composition contains between 0 and about 50 wt %, in particular about 0.1 to about 30.0 wt %, of at least one particulate thickener as the auxiliary, relative to the total weight of the powdery composition. Thickeners are substances that take up liquids, thereby swelling up and transforming into viscous solutions (real solution or gel).

Particularly preferred powdery compositions comprise at least one anionic polymer and/or at least one non-ionic polymer as the particulate thickener. An anionic polymer of this type is preferably selected from among carboxylated celluloses, in particular carboxymethyl cellulose, as the particulate thickener. Polymers with an ethylenic polymer backbone and carboxylic groups are also suitable. Polyacrylic acid is particularly suitable.

In an exemplary embodiment, a suitable particulate thickener is at least one amphiphilic, anionic polymer, containing at least one structural unit of the Formula (V−1) and at least one structural unit of the Formula (V-2),

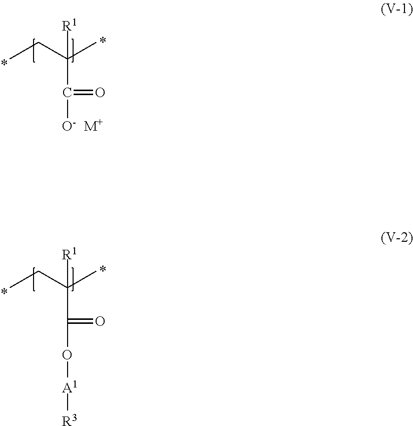

in which
R¹ and R² independently of one another stand for a hydrogen atom or a methyl group,
R³ stands for a (C₈ to C₃₀) alkyl group,
M⁺stands for a physiologically acceptable cation and
A¹ stands for a group *—(CH₂CH₂O)ₓ—* in which x stands for a whole number from 5 to 35, a group *—(CH₂CHMeO)_{y}—* in which y stands for a whole number from 5 to 35 or a group *—(CH₂CH₂O)ₓ—(CH₂CHMeO)_{y}—* in which the sum of x+y stands for a whole number from 5 to 35 and x and y are greater than zero.

Moreover, it is particularly preferred to select the amphiphilic, anionic polymer from at least one polymer, containing at least one structural unit of Formula (V−1) and at least one structural unit of Formula (V-2-a) and at least one structural unit of Formula (V-3),

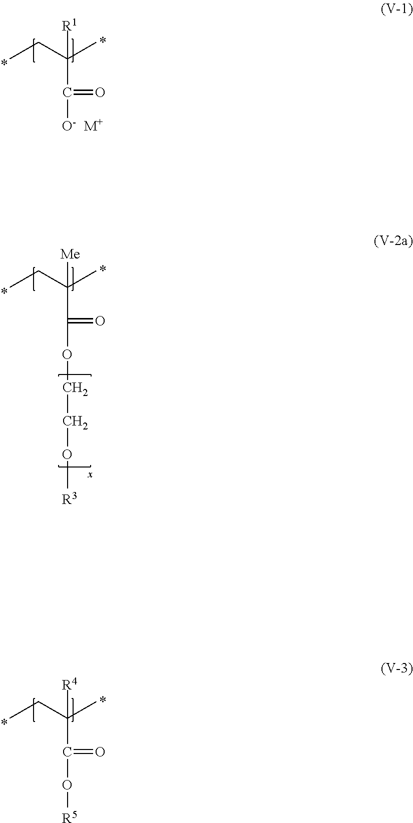

in which
R¹ and R⁴ independently of one another stand for a hydrogen atom or a methyl group,
R³ stands for a (C₈ to C₃₀) alkyl group,
R⁵ stands for a (C₂ to C₄) alkyl group, especially for ethyl,
M⁺stands for a physiologically acceptable cation and
x stands for a whole number from 5 to 35, especially for a whole number from 10 to 30.

A particularly preferred powdery thickener is a crosslinked, amphiphilic, anionic polymer that falls under the NCI name Acrylates/Steareth-20 Methacrylate Crosspolymer. It possesses 20 ethylene oxide units (x according to Formula (ll-a)=20) and is etherified with stearyl alcohol (R³ according to Formula (ll-a)=stearyl). Polymers of this type are marketed for example under the trade name Aculyn® 88 by Rohm & Haas in the form of a 28 to 30 wt % conc. dispersion in water.

A particularly preferred powdery thickener is a crosslinked, amphiphilic, anionic polymer that falls under the NCI name Acrylates/Steareth-20 Methacrylate Crosspolymer. It possesses 20 ethylene oxide units (x according to Formula (ll-a)=20) and is etherified with stearyl alcohol (R³ according to Formula (ll-a)=stearyl). Polymers of this type are marketed for example under the trade name Aculyn® 22 by Rohm & Haas in the form of a 28 to 30 wt % conc. dispersion in water.

A quite particularly preferred amphiphilic, anionic polymer possesses 25 EO units, (x in Formula (V-2a)=25), is etherified with behenyl alcohol (R³ in Formula (V-2a)=C₂₀ alkyl) and is named according to the INCI nomenclature as Acrylates/Beheneth-25 Methacrylate Copolymer. A polymer of this type is available for example under the trade name Aculyn® 28 (Rohm & Haas).

A quite particularly preferred amphiphilic, anionic polymer possesses 25 ethylene oxide units (x in Formula (V-2a)=25), is etherified with palm fatty alcohol (R³ in Formula (IV-a)=alkyl chain distribution of the palm oil fatty acids) and is named according to the NCI nomenclature as Acrylates/Palmeth-25 Acrylate Copolymer. A polymer of this type is available for example from the 3 V Sigma company under the trade name Synthalen® W 2000 as a 30 to 32 wt % conc. emulsion in water.

A preferred particulate thickener useful in the powdery composition contemplated herein is also at least one amphiphilic anionic polymer that is formed from at least one monomer of the Formula (C1), as well as at least one itaconic acid ester monomer of the Formula (C2),

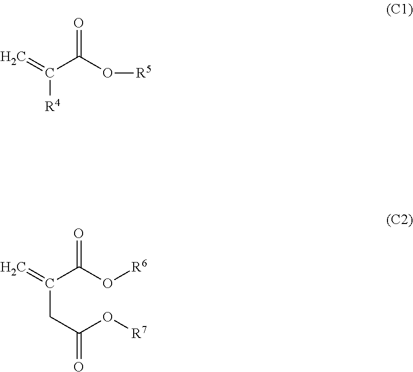

wherein
R⁴ stands for a hydrogen atom or a methyl group,
R⁵ stands for a hydrogen atom or a (C₁ to C₄) alkyl group,
at least one R residue of R⁶ and R⁷ stands for a group -A¹-R⁸ and the respective other for a hydrogen atom, in which
A¹ stands for a group *—(CH₂CH₂O)ₓ—*, in which x stands for a whole number from 5 to 35 (especially for a whole number from 10 to 30),
R⁸ stands for a (C₆ to C₃₀) alkyl group,
M⁺stands for one equivalent of a physiologically acceptable cation.

These amphiphilic anionic polymers are again preferably selected from the group that consists of Acrylates/Ceteth-20 Itaconate Copolymer (e.g. available as Structure® 3001 from Akzo Nobel), Acrylates/Palmeth-25 Itaconate Copolymer (e.g. available as Polygel W30® or Polygel W40® from 3V Sigma), Acrylates/Steareth-20 Itaconate Copolymer (e.g. available as Structure® 2001 from Akzo Nobel).

Quite particularly preferred agents contemplated herein additionally comprise as the powdery thickener at least one sulfonic acid polymer containing at least one structural unit according to Formula (P-I) and at least one structural unit according to Formula (P-II)

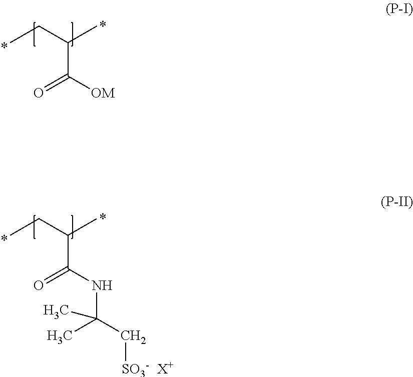

in which M stands for hydrogen, one equivalent of a physiologically acceptable cation (especially a sodium ion) or a (C₂ to C₄) hydroxyalkyl group (in particular 2-hydroxyethyl) and
X⁺stands for a physiologically acceptable cation.
The structural unit of the Formula (P-II) derives from 2-methyl-2[(1-oxo-2-propenyl)amino]-1-propane sulfonic acid (AMPS) that may be partially or completely neutralized. Na⁺and NH₄⁺are usually preferred as the cations.

Preferred sulfonic acid polymers, in which M of the Formula (P-I) stands for hydrogen or sodium, can be described by the general Formula (P1)

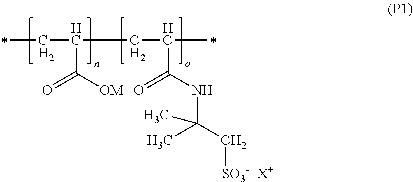

wherein each of the indices n and o vary according to the molecular mass of the polymer and are not intended to necessarily portray block copolymers. In fact, structural units of the Formulas (P-I) and (P-II) can also be statistically distributed in the molecule.

Preferred sulfonic acid polymers of this type are copolymers of 2-methyl-2[(1-oxo-2-propenyl)amino]-1-propane sulfonic acid (AMPS) and sodium acrylate, which are available for example as the commercial product Simulgel® EG (NCI name: Sodium Acrylates/Sodium Acryloyldimethyl Taurate Copolymer, Isohexadecane, Polysorbate 80) from the Seppic company.

In an exemplary embodiment, the additional sulfonic acid polymer contains at least one structural unit of the Formula (P-III) in addition to the structural units of the Formula (P-I) and (P-II)

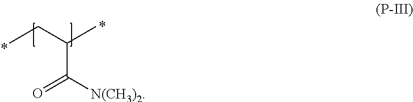

This third monomer is dimethylacrylamide.

Preferred sulfonic acid polymers of this type are copolymers of 2-methyl-2[(1-oxo-2-propenyl)amino]-1-propane sulfonic acid, N,N-dimethylacrylamide and sodium acrylate. Such anionic polymers are marketed as thickeners with the INCI name Sodium Acrylate/Acryloyldimethyltaurate/Dimethylacrylamide Crosspolymer for example under the trade names Sepinov P88 from SEPPIC in the form of a powder.

These preferred sulfonic acid polymers can be described by the general Formula (P2)

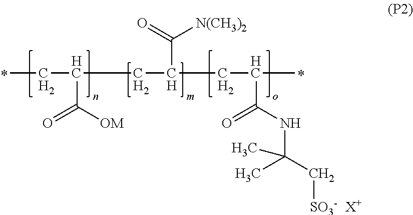

wherein each of the indices m and n and o vary according to the molecular mass of the polymer and are not intended to necessarily portray block copolymers. In fact, structural units of the Formulas (P-I), (P-II) and (P-III) can also be statistically distributed in the molecule.

The sulfonic acid polymer preferably comprises the monomers of the Formulas (P-I), (P-II) and (P-III) in such a number and distribution that the copolymer A has molecular masses between suitable 5 and suitable 1000 kDa. The sulfonic acid polymer preferably comprises the monomers of the Formulas (P-I), (P-II) and (P-III) within specific limits. Here, preferred agents are characterized in that they comprise sulfonic acid polymers that comprise
suitable 10 to suitable 90 mol %, preferably suitable 15 to suitable 85 mol % and especially suitable 20 to suitable 80 mol % of monomers of the Formula (P-I) and
suitable 5 to suitable 85 mol %, preferably suitable 7.5 to suitable 80 mol % and especially suitable 10 to suitable 60 mol % of monomers of the Formula (P-II) and
suitable 5 to suitable 85 mol %, preferably suitable 10 to suitable 80 mol % and especially suitable 15 to suitable 70 mol % of monomers of the Formula (P-III).

If the residue R in the above formula (P-I) stands for a (C₂ to C₄) hydroxyalkyl group, in particular for a 2-hydroxyethyl group, then the resulting anionic polymers are the most preferred powdery thickeners, from which at least one compound is contained in the powdery compositions contemplated herein. Anionic polymers of this embodiment are marketed with the INCI name Hydroxyethylacrylate/Sodium Acryloyldimethyl Taurate Copolymer for example in powder form from Seppic under the trade name Sepinov® EMT 10. Independently of whether the agents contemplated herein comprise one or more sulfonic acid polymers, it is preferred to incorporate the sulfonic acid polymers within specific quantity ranges. Here, preferred powdery agents contain the sulfonic acid polymer(s) in an amount of suitable 0.1 to suitable 50 wt %, preferably suitable 5.0 to suitable 40.0 wt % and particularly suitable 10.0 to suitable 30.0 wt %, each relative to the weight of the agent.

In the context of another embodiment, the powdery compositions comprise such particles (in particular, selected from at least one anionic polymer and/or at least one non-ionic polymer) whose average particle diameter (d0.5 volume-related, determined by means of laser diffraction) is in a range of about 10 μm to about 800 μm, in particular about 25 μm to about 500 μm.

The following embodiments (A) to (J) are quite particularly preferred in the context of the invention:

(A): A powdery composition, comprising, relative to its weight
(a) about 5 to about 100 wt % of at least one powdery compound that is effective as a hair cosmetic, selected from at least one representative of the group that consists of solid, film-forming compounds and surfactants,
(b) 0 to about 25 wt % of at least one powdery solid as a filler which differs from (a) and (c),
(c) between 0 and about 50 wt %, in particular from about 0.1 to about 30 wt % of at least one auxiliary that differs from (a) and (b) and is selected from emulsifiers and thickeners as well as their mixtures,
   with the proviso that about 0.4 g of the powdery composition dissolves in 100 mL water at 35° C. within not more than about 5 minutes, in particular within not more than about 4 minutes, when mixed in a tumble mixer at 101 revolutions per minute.

(B): A powdery composition, comprising, relative to its weight
(a) about 5 to 100 wt % of at least one powdery compound that is effective as a hair cosmetic, selected from at least one representative of the group that consists of setting polymers and waxes,
(b) 0 to about 25 wt % of at least one powdery solid as a filler which differs from (a) and (c),
(c) between 0 and about 50 wt %, in particular from about 0.1 to about 30 wt % of at least one auxiliary that differs from (a) and (b) and is selected from emulsifiers and thickeners as well as their mixtures,
   with the proviso that about 0.4 g of the powdery composition dissolves in 100 mL water at 35° C. within not more than about 5 minutes, in particular within not more than about 4 minutes, when mixed in a tumble mixer at 101 revolutions per minute.

(C): A powdery composition, comprising, relative to its weight
(a) about 5 to 100 wt % of at least one powdery compound that is effective as a hair cosmetic, selected from at least one representative of the group that consists of setting polymers and waxes,
(b) 0 to about 25 wt % of at least one powdery solid as a filler which differs from (a) and (c),

(c) between 0 and about 50 wt %, in particular from about 0.1 to about 30 wt % of at least one auxiliary that differs from (a) and (b) and is selected from at least one powdery thickener selected from the group that consists of anionic polymers and non-ionic polymers,

with the proviso that about 0.4 g of the powdery composition dissolves in 100 mL water at 35° C. within not more than about 5 minutes, in particular within not more than about 4 minutes, when mixed in a tumble mixer at 101 revolutions per minute.

(D): A powdery composition, comprising, relative to its weight
(a) about 5 to about 100 wt % of at least one powdery compound that is effective as a hair cosmetic, selected from at least one representative of the group that consists of setting polymers and waxes,
(b) 0 to about 25 wt % of at least one powdery solid as a filler which differs from (a) and (c),
(c) between 0 and about 50 wt %, in particular from about 0.1 to about 30 wt % of at least one auxiliary that differs from (a) and (b) and is selected from at least one powdery thickener selected from sulfonic acid polymers containing at least one structural unit according to Formula (P-I) and at least one structural unit according to Formula (P-II)

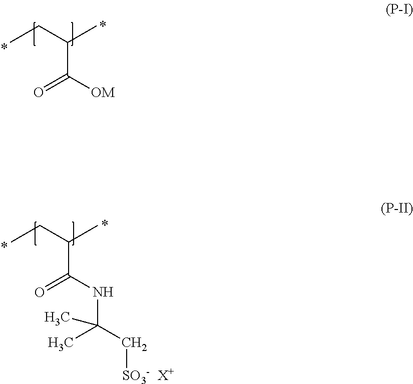

in which M stands for hydrogen, one equivalent of a physiologically acceptable cation (especially a sodium ion) or a (C₂ to C₄) hydroxyalkyl group (in particular 2-hydroxyethyl) and
X⁺stands for a physiologically acceptable cation,
with the proviso that about 0.4 g of the powdery composition dissolves in 100 mL water at 35° C. within not more than about 5 minutes, in particular within not more than about 4 minutes, when mixed in a tumble mixer at 101 revolutions per minute.

(E): A powdery composition, comprising, relative to its weight
(a) about 5 to 100 wt % of at least one powdery compound, effective as a hair cosmetic agent, selected from at least one wax,
(b) 0 to about 25 wt % of at least one powdery solid as a filler which differs from (a) and (c),
(c) between 0 and about 50 wt %, in particular from about 0.1 to about 30 wt % of at least one auxiliary that differs from (a) and (b) and is selected from at least one emulsifiers and optionally from at least one thickener,
   with the proviso that about 0.4 g of the powdery composition dissolves in 100 mL water at 35° C. within not more than about 5 minutes, in particular within not more than about 4 minutes, when mixed in a tumble mixer at 101 revolutions per minute.

F): A powdery composition, comprising, relative to its weight
(a) about 5 to about 100 wt % of at least one powdery compound, effective as a hair cosmetic agent, selected from at least one wax,
(b) 0 to about 25 wt % of at least one powdery solid as a filler which differs from (a) and (c),
(c) between 0 and about 50 wt %, in particular from about 0.1 to about 30 wt % of at least one auxiliary that differs from (a) and (b) and is selected from at least one emulsifier and optionally from at least one powdery thickener selected from the group that consists of anionic polymers and non-ionic polymers,
   with the proviso that about 0.4 g of the powdery composition dissolves in 100 mL water at 35° C. within not more than about 5 minutes, in particular within not more than about 4 minutes, when mixed in a tumble mixer at 101 revolutions per minute.

(G): A powdery composition, comprising, relative to its weight
(a) about 5 to about 100 wt % of at least one powdery compound, effective as a hair cosmetic agent, selected from at least one wax,
(b) 0 to about 25 wt % of at least one powdery solid as a filler which differs from (a) and (c),
(c) between 0 and about 50 wt %, in particular from about 0.1 to about 30 wt % of at least one auxiliary that differs from (a) and (b) and is selected from at least one emulsifier and optionally powdery thickener selected from sulfonic acid polymers containing at least one structural unit according to Formula (P-I) and at least one structural unit according to Formula (P-II)

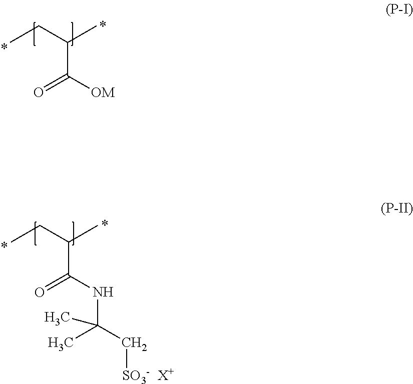

in which M stands for hydrogen, one equivalent of a physiologically acceptable cation (especially a sodium ion) or a (C₂ to C₄) hydroxyalkyl group (in particular 2-hydroxyethyl) and

X⁺stands for a physiologically acceptable cation,

with the proviso that about 0.4 g of the powdery composition dissolves in 100 mL water at 35° C. within not more than about 5 minutes, in particular within not more than about 4 minutes, when mixed in a tumble mixer at 101 revolutions per minute.

(H): A powdery composition, comprising, relative to its weight
(a) about 5 to 100 wt % of at least one powdery compound, effective as a hair cosmetic agent, selected from at least one setting polymer,
(b) 0 to about 25 wt % of at least one powdery solid as a filler which differs from (a) and (c),
(c) between 0 and about 50 wt %, in particular from about 0.1 to about 30 wt % of at least one auxiliary that differs from (a) and (b) and is selected from at least one thickener and optionally additionally from at least one emulsifier,
   with the proviso that about 0.4 g of the powdery composition dissolves in 100 mL water at 35° C. within not more than about 5 minutes, in particular within not more than about 4 minutes, when mixed in a tumble mixer at 101 revolutions per minute.

(I): A powdery composition, comprising, relative to its weight
(a) about 5 to 100 wt % of at least one powdery compound, effective as a hair cosmetic agent, selected from at least one setting polymer,
(b) 0 to about 25 wt % of at least one powdery solid as a filler which differs from (a) and (c),
(c) between 0 and about 50 wt %, in particular from about 0.1 to about 30 wt % of at least one auxiliary that differs from (a) and (b) and is selected from at least one powdery thickener from the group that consists of anionic polymers and non-ionic polymers, and optionally additionally from at least one emulsifier,
   with the proviso that about 0.4 g of the powdery composition dissolves in 100 mL water at 35° C. within not more than about 5 minutes, in particular within not more than about 4 minutes, when mixed in a tumble mixer at 101 revolutions per minute.

(J): A powdery composition, comprising, relative to its weight
(a) about 5 to about 100 wt % of at least one powdery compound, effective as a hair cosmetic agent, selected from at least one setting polymer,
(b) 0 to about 25 wt % of at least one powdery solid as a filler which differs from (a) and (c),
(c) between 0 and about 50 wt %, in particular from about 0.1 to about 30 wt % of at least one auxiliary that differs from (a) and (b) and is selected from at least one powdery thickener selected from sulfonic acid polymers, containing at least one structural unit according to Formula (P-I) and at least one structural unit according to Formula (P-II)

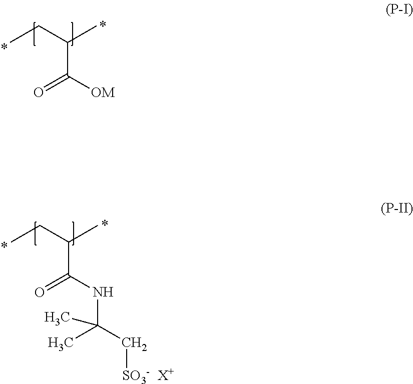

in which M stands for hydrogen, one equivalent of a physiologically acceptable cation (especially a sodium ion) or a (C₂ to C₄) hydroxyalkyl group (in particular 2-hydroxyethyl) and
X⁺stands for a physiologically acceptable cation.
   with the proviso that about 0.4 g of the powdery composition dissolves in 100 mL water at 35° C. within not more than about 5 minutes, in particular within not more than about 4 minutes, when mixed in a tumble mixer at 101 revolutions per minute.

For the embodiments (A) to (J) the previously preferred embodiments characterized by the features stated therein (vide supra) likewise apply mutatis mutandis as preferred. Furthermore, the average particle diameters stated as preferred are preferred in the context of the embodiments (A) to (J).

The powdery compositions contemplated herein can be presented in almost any containers. It only has to be ensured that the powder is not too highly mechanically stressed when the composition is withdrawnso that the powder is not already converted into liquid form when it is withdrawn. For example, pots, bottles and also tetrapacks are suitable, wherein the container can be equipped for example with a pouring and metering device.

In another exemplary embodiment, the powdery compositions contemplated herein can be prepared by the following preferred production process.

In a further exemplary embodiment, the powdered composition contemplated herein is uded for the temporary shaping of keratin-containing fibres, in particular human hair.

Yet another exemplary embodiment provides for a method for the cosmetic treatment of human hair in which method
(i) a surface used as the application aid is moistened,
(ii) a powdery composition as contemplated herein is applied onto the moistened surface of the application aid,
(iii) the hair is brought into contact with the surface from (ii) for the application of the powdery composition, effective as a hair cosmetic agent, and arranged for the hairstyle, and
(iv) the hair is dried,
   with the proviso that the hair is not rinsed after step (iii).

Exemplary application aids for applying the powdery composition are the hand, a brush, a sponge, a cloth, a hairbrush, a boar brush, a hair roller, a mascara brush or a comb. In this regard, it is preferred if the surface of the application aid that is used is the palm of the hand.

The bringing into contact according to step (iii) is for example a deliberate massaging of the powder into the hair, combing or brushing.

The hair styling according to step (iii) can be effected in one operation by applying said at least one powdery compound that is effective as a hair cosmetic.

The drying in step (iv) can be effected by drying in ambient air or e.g. by warm air from a hairdryer.

A further exemplary embodiment provides for a method for the cosmetic treatment of human hair in which method

(i) the hair is moistened,
(ii) a powdery composition as contemplated herein is applied onto the moistened hair,
(iii) the hair is styled, and
(iv) the hair is dried,
with the proviso that the hair is not rinsed after step (ii).

The moistening of the hair in step (i) can be effected by spraying the fibers with a liquid, preferably with water. Preferably, in step (i) the fibers are shampooed with a conventional shampoo, rinsed and then rubbed with a towel. At the conclusion of the rubbing step the hair is not dripping wet, but remains noticeably damp. The powdery composition is applied onto the hair in step (ii) for example by means of a type of salt cellar dosing system.

The hair styling according to step (iii) can be effected in one operation by applying the powdery compound that is effective as a hair cosmetic. The drying in step (iv) can be effected by drying in ambient air or, e.g., by warm air from a hairdryer.

### Examples

### 1. Production of the Powdery Compositions

The inventive powdery compositions E1 to E13 were produced as described below with the following compositions:

| Raw material | E1 | E2 | E3 | E4 | E5 | E6 | E7 | E8 |
|---|---|---|---|---|---|---|---|---|
| Sepinov ® | 90.0 | 90.0 | 90.0 | 90.0 | 15.0 | 20.0 | 15.0 | 17.5 |
| EMT, powder | | | | | | | | |
| Celquat ® | 10.0 | — | — | — | — | — | — | — |
| L-200, powder | | | | | | | | |
| Polyvinyl | — | 10.0 | — | — | — | — | 25.0 | — |
| pyrrolidone, | | | | | | | | |
| powder | | | | | | | | |
| Vinyl alcohol/ | — | — | 10.0 | — | — | — | — | — |
| vinyl formate | | | | | | | | |
| copolymer, | | | | | | | | |
| powder | | | | | | | | |
| Advantage ® | — | — | — | 10.0 | 85.0 | 80.0 | 60.0 | 81.9 |
| S, powder | | | | | | | | |
| Perfume | — | — | — | — | — | — | — | 0.6 |
| Dissolution rate | 240 | 220 | 270 | 250 | 250 | 210 | 220 | 210 |
| in seconds | | | | | | | | |

| Raw material | E9 | E10 | E11 | E12 | E13 |
|---|---|---|---|---|---|
| Sepinov ® | 17.5 | 20.0 | 22.5 | 20.0 | 20.0 |
| EMT 10, powder | | | | | |
| Advantage ® S, | 82.0 | 79.5 | 77.0 | 78.8 | 78.8 |
| powder | | | | | |
| Perfume | 0.5 | 0.5 | 0.5 | 1.2 | 1.2 |
| Dissolution rate | 210 | 210 | 210 | 210 | 210 |
| in seconds | | | | | |

All powdery raw materials were individually ground in a grinder. The raw materials were appropriately mixed. The thus-obtained finished styling powder was filled in polyethylene bottles. The dissolution rate was determined following the previously described test method (vide supra).

2.0 Application

2.1 On Moist Hair

Thirteen strands of hair were treated with one of each of the above styling agents. For this the strand of hair was moistened and the inventive agent was applied and massaged into the moist strand. The treated strand of hair was stretched and fixed on a board and allowed to dry.

The strands with the inventive compositions E1 to E13 had an outstanding shape retention. The hair had clearly more structure and texture. In spite of the added particulate raw materials no visible matting of the hair was observed. The hair retained its natural gloss.

2.2 On Dry Hair.

Thirteen strands of hair were treated with the hand with one of each of the above styling agents. For this the palm of the hand was moistened with water and the inventive agent was rubbed onto the moist palm of the hand to form a gel. The resulting gel was applied onto the strands of hair and massaged in. The treated strand of hair was stretched and fixed on a board and allowed to dry.

The hair had clearly more structure and texture. In spite of the added particulate raw materials no visible matting of the hair was observed. The hair retained its natural gloss.

3. Index of the employed raw materials:

The raw materials employed in the examples are defined as follows:

| | |
|---|---|
| Sepinov EMT 10, powder | copolymer of 2-hydroxyethyl acrylate and 2- |
| | methyl-2-(1-oxo-2-propenyl)amino)-1-propane |
| | sulfonic acid monosodium salt (INCI name: |
| | Hydroxyethyl acrylate/Sodium acryloyl |
| | dimethyl Taurate Copolymer) (Seppic) |
| Celquat L-200, powder | quaternized cellulose derivative (INCI name: |
| | Polyquaternium-4) (National Starch) |
| Advantage S, powder | vinyl caprolactam/vinyl pyrrolidone/ |
| | dimethylaminoethyl methacrylate copolymer |
| | (INCI name: Vinyl Caprolactam/VP/ |
| | Dimethylaminoethyl Methacrylate |
| | Copolymer) (ISP). |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

1. A powdery composition comprising, relative to its total weight:
(a) about 5 to 100 wt % of at least one powdery compound, effective as a hair cosmetic agent;
(b) 0 to about 25 wt % of at least one powdery solid as a filler which differs from (a) and (c);
(c) 0 to about 95 wt % of at least one auxiliary that differs from (a) and (b) and is selected from emulsifiers and thickeners as well as their mixtures,
with the proviso that about 0.4 g of the powdery composition dissolves in 100 mL water at 35° C. within not more than about 5 minutes when mixed in a tumble mixer at 101 revolutions per minute.

2. The powdery composition according to claim 1, wherein not more than 8 wt % liquid is present in the powdery composition relative to the total weight of the powdery composition.

3. The powdery composition according to claim 1, wherein the at least one powdery compound is chosen from solid, film-forming compounds.

4. The powdery composition according to claim 3, wherein the solid, film-forming compounds are selected from setting polymers, waxes and film-forming polymers.

5. The powdery composition according to claim 1, wherein the auxiliary comprises in the range of 0 to about 50 wt % of a particulate thickener, relative to the total weight of the powdery composition.

6. The powdery composition according to claim 5, wherein the particulate thickener comprises an anionic polymer and/or a non-ionic polymer.

7. The powdery composition according to claim 1, wherein a volume-related average particle diameter (d0.5) of the powdery composition is in a range of about 10 μm to about 800 μm.

8. A method for the cosmetic treatment of human hair, the method comprising the steps of:
(i) moistening the hair;
(ii) applying to the hair a powdery composition, the powdery composition comprising relative to its total weight:
(a) about 5 to 100 wt % of at least one powdery compound, effective as a hair cosmetic agent;
(b) 0 to about 25 wt % of at least one powdery solid as a filler which differs from (a) and (c);
(c) 0 to about 95 wt % of at least one auxiliary that differs from (a) and (b) and is selected from emulsifiers and thickeners as well as their mixtures,
with the proviso that about 0.4 g of the powdery composition dissolves in 100 mL water at 35° C. within not more than about 5 minutes when mixed in a tumble mixer at 101 revolutions per minute;
(iii) mechanically stressing the hair; and
(iv) drying the hair,
with the proviso that the hair is not rinsed after step (ii).

9. A method for the cosmetic treatment of human hair, the method comprising the steps of:
(i) moistening a surface used as an application aid;
(ii) applying onto the moistened surface of the application aid; a powdery composition comprising relative to its total weight:
(a) about 5 to 100 wt % of at least one powdery compound, effective as a hair cosmetic agent;
(b) 0 to about 25 wt % of at least one powdery solid as a filler which differs from (a) and (c);
(c) 0 to about 95 wt % of at least one auxiliary that differs from (a) and (b) and is selected from emulsifiers and thickeners as well as their mixtures,
with the proviso that about 0.4 g of the powdery composition dissolves in 100 mL water at 35° C. within not more than about 5 minutes when mixed in a tumble mixer at 101 revolutions per minute;
(iii) contacting the hair with the surface from (ii) for the application of the powdery compound, effective as a hair cosmetic agent, and arranging for a hairstyle, and
(iv) drying the hair,
with the proviso that the hair is not rinsed after step (iii).

10. The method according to claim 9, wherein the surface used as the application aid is the palm of the hand.

11. The powdery composition according to claim 2, wherein not more than 5 wt % of a liquid is present in the powdery composition relative to the total weight of the powdery composition.

12. The powdery composition according to claim 4, wherein the solid, film-forming compounds are selected from setting polymers and waxes.

13. The powdery composition according to claim 7, wherein a volume-related average particle diameter (d0.5) of the powdery composition is in a range of about 25 μm to about 500 μm.
